(19) United States
(12) Patent Application Publication
(10) Pub. No.: US 2005/0163648 A1
(43) Pub. Date: Jul. 28, 2005

(54) METHOD AND APPARATUS FOR STERILIZING AIR IN LARGE VOLUMES BY RADIATION OF ULTRAVIOLET RAYS

(76) Inventor: MICHAEL Y. LIANG, ALAMEDA, CA (US)

Correspondence Address: MICHAEL Y. LIANG, 218 CREEDON CIRCLE, ALAMEDA, CA, 94502, US

(21) Appl. No.: 10/707,919

(22) Filed: Jan. 26, 2004

(51) Int. Cl. A61L 2/00; A62B 7/08; B01J 19/08; A61L 9/00

(52) U.S. Cl. 422/1; 422/24; 422/121; 422/186; 422/305

(57) ABSTRACT

A sterilizing method and apparatus for destroying live microorganisms including viruses in fluent material, such as air and water, in large volume. As a preferred embodiment of this invention, air is drawn, from the inlet 1, through the inlet filter unit 3 and into the circuitous sterilizing chamber 10 that is irradiated by numbers of UV lamp tubes 15, and through the discharging filter unit 13 and then out through outlet 11. A sample window 12 is built in for quality supervision. Visual inspection windows 5 and UV sensors 6 are provided for easy supervision and auto-control. The sterilizing chamber 10 also includes flow resistant reducing feature 7 and internal reflecting surfaces 9.

### BACKGROUND OF INVENTION

1. Technical Field

This invention is about an air sterilizing system, which uses intense ultraviolet (UV) irradiation to free air from live bacteria, viruses and other microorganisms. The method can also be applied to sterilize any fluent material, including gas, water or other fluids, containing every kind of live microorganisms naturally with or from biological agents used by terrorists or in warfare.

2. Background

The air transmission of harmful bacteria, viruses and other microorganisms is one of the most common causes of infectious disease in the world today. The deaths related to contaminated air are countless. Only influenza kills more than forty thousand people every year.

The worldwide outbreak of SARS (caused by coronaviruses) has become a serious global concern since Jan. 2003. According to WHO, the amplification of transmission within well-equipped hospitals was a striking feature of SARS. In some cases, staff became infected despite wearing full protective equipment. Thus, although SARS is not thought to be an airborne infection, a disproportionately large amount of resources is needed to prevent transmission of airborne infections as compelling evidence states that aerosols and microdroplets with coronaviruses in air may play very important roles in SARS transmission. In the same way, many non-airborne harmful bacteria and viruses can become airborne when they are in the form of aerosols or microdroplets.

Further, the modern air-conditioned buildings make microorganisms, including viruses, spread out to thousands of people easily and fast. In order to save energy, the buildings reuse most of the used air with only 10% to 20% fresh in. By using return air ducts, contaminated air that contains all respiratory microdroplets with million kinds of bacteria and viruses and toxic chemical gases emitted from building furnishings and consumer products, mixes together, like a cocktail, then is distributed to all people in the buildings. Every year, there are more than ten of thousand deaths with countless illnesses related to poor conditioned air in the world.

Furthermore, if there is any terrorist distributing harmful biological agents, such as Bacillus Anthracis, in the form of aerosols, via air conditioning systems of big buildings, it will kill many people.

3. Prior Art

For preventing the air transmission of disease, many air purification devices have been created and patented. But none of them was created for sterilizing air in large volumes and destroying more than 99.999% of the microorganisms in the air. For example, U.S. Pat. Nos. 4,118,191, 4,210,429, 4,786,812, 4,806,768, 4,917,713, 4,931,654, 4,990,311, 5,185,015, 6,264,888, 6,464,760, 6,497,840, all these were designed with some or all of following components: blower, filter(s) and a few UV lamps, as small devices for use in rooms. All of them have a so short sterilizing path or a so small chamber that the sterilizing effect is quite questionable. The weakest point of such apparatus is that they do not offer enough dosage of UV radiation to kill microorganisms. On the contrary, most of the above devices may have dangerous effects that they actually aerosolize microorganisms and blow them all over.

Another approach is the sterilization of the cooling coil, filters or other parts of an air conditioning system by radiation of UV rays, such as U.S. Pat. Nos. 4,990,313, 5,225,167. They can destroy bacterial accumulations on those parts. However, the filters do not catch all of the microorganisms. The latest filtration systems can only filter air on 0.12 micrometer (120 nm) size particles. But the diameter of bacteria and viruses are usually smaller than 0.12 micrometer. Other weakness of such systems is the high cost in purchase, use and maintenance.

As other inventions like patent Publication No. U.S. 2003/0086848, they are mostly created for liquids. Neither can they solve the above-mentioned problems.

### SUMMARY OF INVENTION

This invention is about an air sterilizing method and apparatus to destroy all live microorganisms in the air in large volumes to satisfy the increasing needs for the purposes of anti infectious disease and anti-terrorism. These apparatus can sterilize either fresh air or return air before distribution. Or they can be used to sterilize contaminated air before exhausting it to the environment. An apparatus can be designed for a killing rate higher than 99.999% by adjusting the number of UV lamps and extending the length of the circuitous sterilizing chamber(s). The employment of circuitous chamber(s) is for the purpose of increasing exposure to UV radiation that is used to kill all live microorganisms that pass through the chamber.

Since UV radiation at about 253.7 nm is very effective in killing microorganisms, the apparatus of this invention are very effective. These apparatus can be added onto existing air conditioning systems, or stand alone, for hospitals, biomedical, pharmaceutical, biotechnology, genetic research, universities, laboratories, food processing, semiconductor fabrication, industrial processing systems, governmental and military buildings, commercial buildings and any public buildings.

This invention can also be applied to devise small sterilizing apparatus for transportation vehicles and residential shelters.

This invention can also be used to sterilizing all kinds of fluent material, especially water.

### BRIEF DESCRIPTION OF DRAWINGS

FIG. 1A fragmentary perspective view of the apparatus for sterilizing air in large volume by radiation of ultraviolet rays according to one preferred embodiment of this invention.

FIG. 2 Depiction of a front elevation view and a top view of the apparatus for sterilizing air in large volume by radiation of ultraviolet rays of FIG. 1.

FIG. 3 Depiction of a front elevation view and a top view of the apparatus for sterilizing air in large volume by radiation of ultraviolet rays with substitution of UV lamps of FIG. 1.

FIG. 4 Depiction of a front elevation view and a top view of the apparatus for sterilizing air in large volume by radiation of ultraviolet rays according to another preferred embodiment of this invention.

FIG. 5 Depiction of a front elevation view and a top view of the apparatus for sterilizing water in large volume by radiation of ultraviolet rays according to a preferred embodiment of this invention.

FIG. 6 Depiction of a front elevation view and a top view of the apparatus for sterilizing water in large volume by radiation of ultraviolet rays according to another preferred embodiment of this invention.

List of reference numbers for the major elements in the drawings.

1 Inlet.

2 Blower, fan, pump or other power unit.

3 Inlet filter unit.

4 UV lamp fixture.

5 UV visual inspection window.

6 UV sensor.

7 Curved flow guiding interior.

8 Housing of the apparatus.

9 Interior mirror surface.

10 Circuitous sterilizing chamber.

11 Outlet.

12 Inspection window or sample faucet.

13 Outlet filter unit.

14 Flow direction.

15 UV lamp tube.

16 Maintenance door.

17 Inlet valve.

18 Outlet valve.

### DETAILED DESCRIPTION

Modes for carrying out the invention.

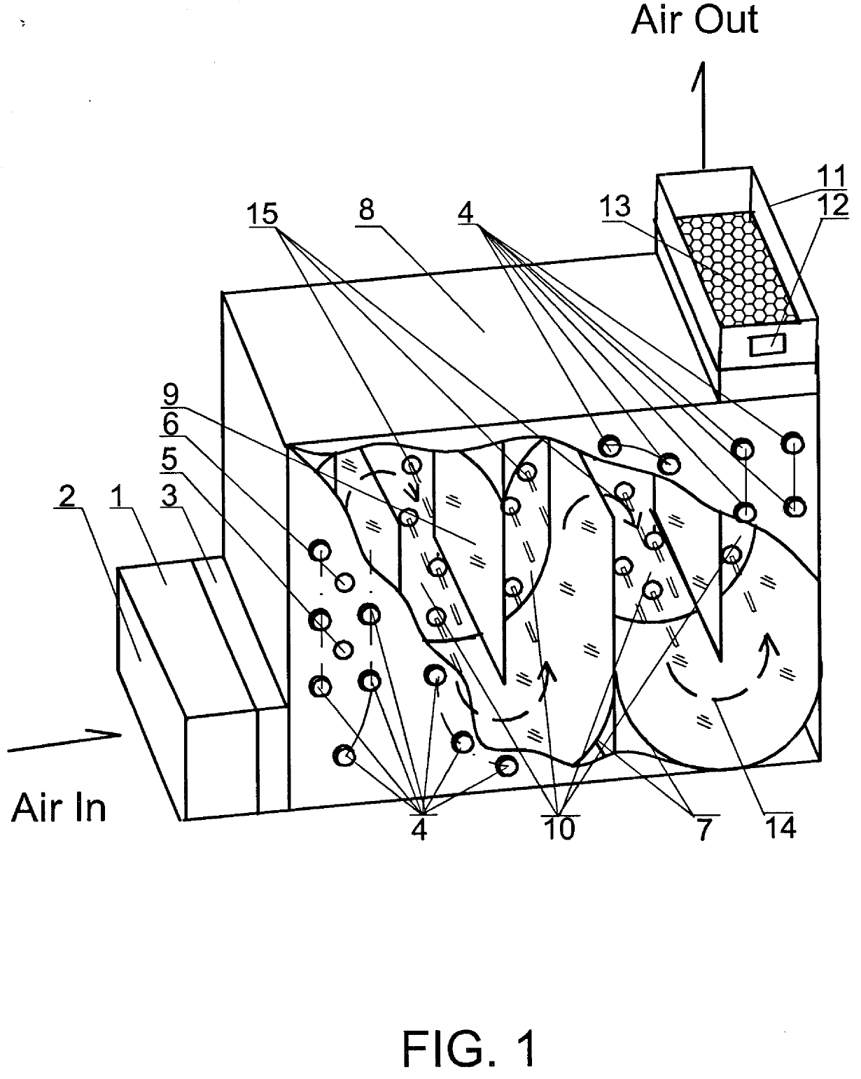

Referring to FIG. 1, the basic construction of an apparatus for sterilizing air in large volume by radiation of ultraviolet rays in accordance with this invention is shown, including an exterior housing 8 with an air Inlet 1, an blower or fan and associated motor 2, an inlet filter unit 3, a roundabout UV germicidal sterilizing chamber 10 with UV visual inspection windows 5 and UV sensors 6 on it, an air outlet 11 with an inspection window 12 and an outlet filter 13.

The air inlet 1 is preferably positioned on the lowest part of the sidewall of the housing. Since air inlet 1 may connect to a fresh and/or return air duct (not shown,) the opening size and shape of the inlet 1 may match the air duct. However, it shall be decided by the volume of air to be sterilized when it is for new installation. Thus, the size of inlet 1 shall be the same as the sterilizing chamber 10 and outlet 11.

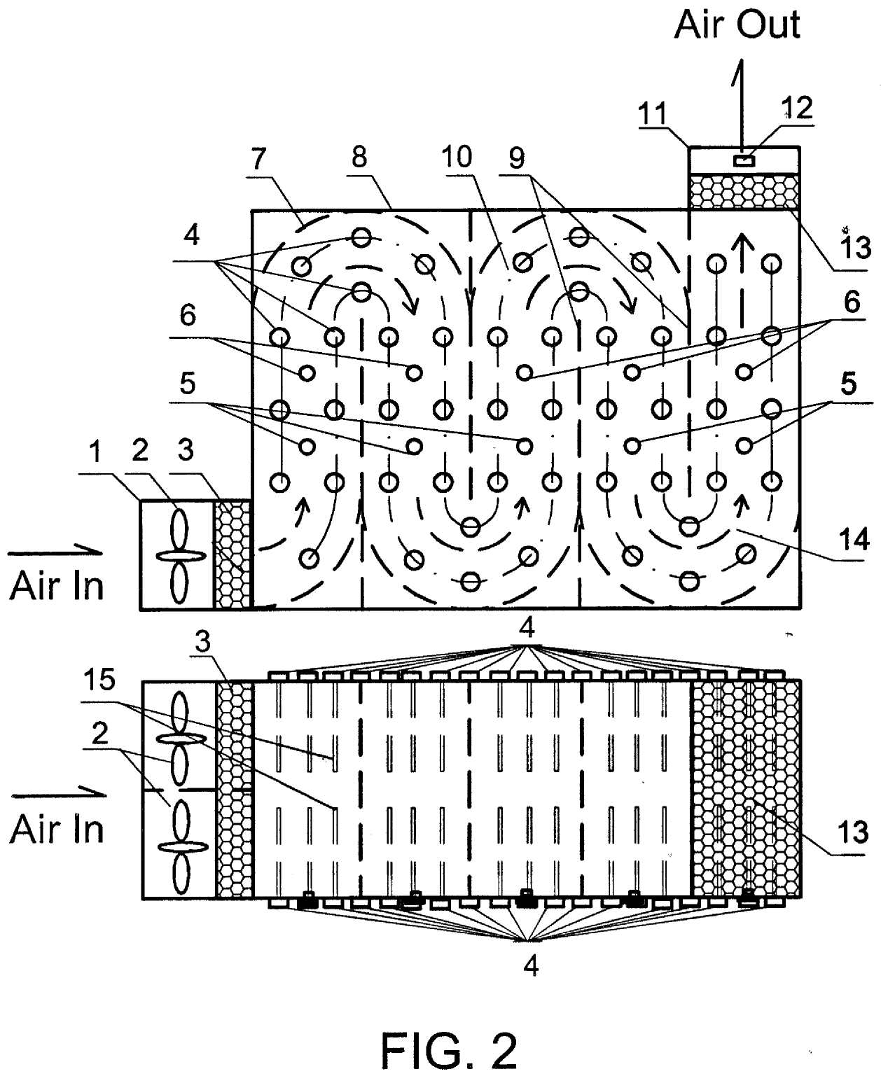

As better shown in FIG. 2, within the inlet 1, there is preferably a blower or fan and associated motor 2 to give air enough power to go though the apparatus. Connected to the inlet 1, there is an inlet filter unit 3 so that all air drawn through the inlet 1 must pass through the inlet filter 3 before entering the chamber 10. The basic function of the inlet filter unit 3 is intercepting and retaining any fairly large particles to protect UV lamp tubes 15 in said chamber 10 where air flows from the inlet filter unit 3 to the outlet filter unit 13.

The air outlet 11 is preferably positioned on the top of the housing 8 so that the sterilized discharging air can easily goes into the air distribution duct (not shown) that leads to every rooms in a building, or into air exhaust pipe(s) (not shown) to the air outside. Between the sterilizing chamber and the outlet, there is an outlet filter unit 13. The purpose of this filter is to prevent particles from getting into the air distribution duct. So, the outlet filter unit 13 can be designed according to the requirements of application, from normal filters to HEPA/ULPA filters, preferably HEPA filters for most of the applications. The outlet filter unit is also comprised of a catalytic filter to convert ozone into oxygen. On the outlet 11, there is an inspection window 12 for taking air samples for live microorganisms inspection to supervise sterilizing effect and air quality.

In the sterilizing chamber 10, which is constructed basically by the six sides of the housing 8, there is always a curved (circular) flow guiding interior 7 to make a smooth roundabout wherever the air flow turns its direction in the chamber 10 to reduce flow resistance. The interior surfaces 9 of the sterilizing chamber 10 is made with antiultraviolet, light reflecting material with mirror surface to increase the interior reflection and thus increase the UV sterilizing effect. The length and/or the number of roundabouts of the chamber 10 can be reduced or increased according with the number of UV lamps to be installed. The opening size of the sterilizing chamber 10 is mainly decided by the volume of air to be sterilized. Normal sizes include, but not limit to, 1′×1′, 1′×2′, 2′×2′, 2′×3′, 3′×3′, 3′×4′, 4′×4′.

Positioned in the circuitous sterilizing chamber 10, are numbers of UV lamp tubes 15. These UV lamps penetrate through the walls of the chamber 10 from outside of the front and back walls of the housing 8. Their fixtures 4 and wirings are fixed outside the housing 8 so that it is easy to perform lamp exchange periodically or other maintenance. In a preferred embodiment of this invention, the UV lamps 15 are preferably single end (but can be double ends) tube-shape non-ozone germicidal lamps that emit UV radiation mainly at about 253.7 nm, which is the most sensitive UV radiation to all microorganisms. For easily supervision UV lamps, there are preferably one UV visual inspection window 5 and one UV sensor 6 in every section the sterilizing chamber 10.

The fundamental difference of this invention from prior art methods and apparatus that were thought having the ability to kill all of microorganisms with only one, two or three UV lamps in a wink, is the UV radiation exposure intensity. The basic formula is that the product (UV radiation value) of UV power multiplying exposure time must be higher than the UV death value of any microorganisms. In other words, the sterilizing dosage of UV radiation should be high enough that there will not be any microorganism survived.

In order to accomplish this goal, a circuitous sterilizing chamber 10, which can increase both the traveling time of the sterilized air and the number of UV lamps installed, is employed. In order to get ideal UV intensity, the distance between any two UV lamps is preferably about 3″ to 1′. They can line up in any pattern. In the preferred embodiment of this invention, there are 98 UV lamp tubes 15 in two rows along the circuitous sterilizing chamber 10. These lamps are fixed on both front and rear side of the chamber 10. Increasing the number of UV lamps can increase the sterilizing power of the apparatus. The length and roundabouts of the chamber 10 can also be increased to get more space to install UV lamps.

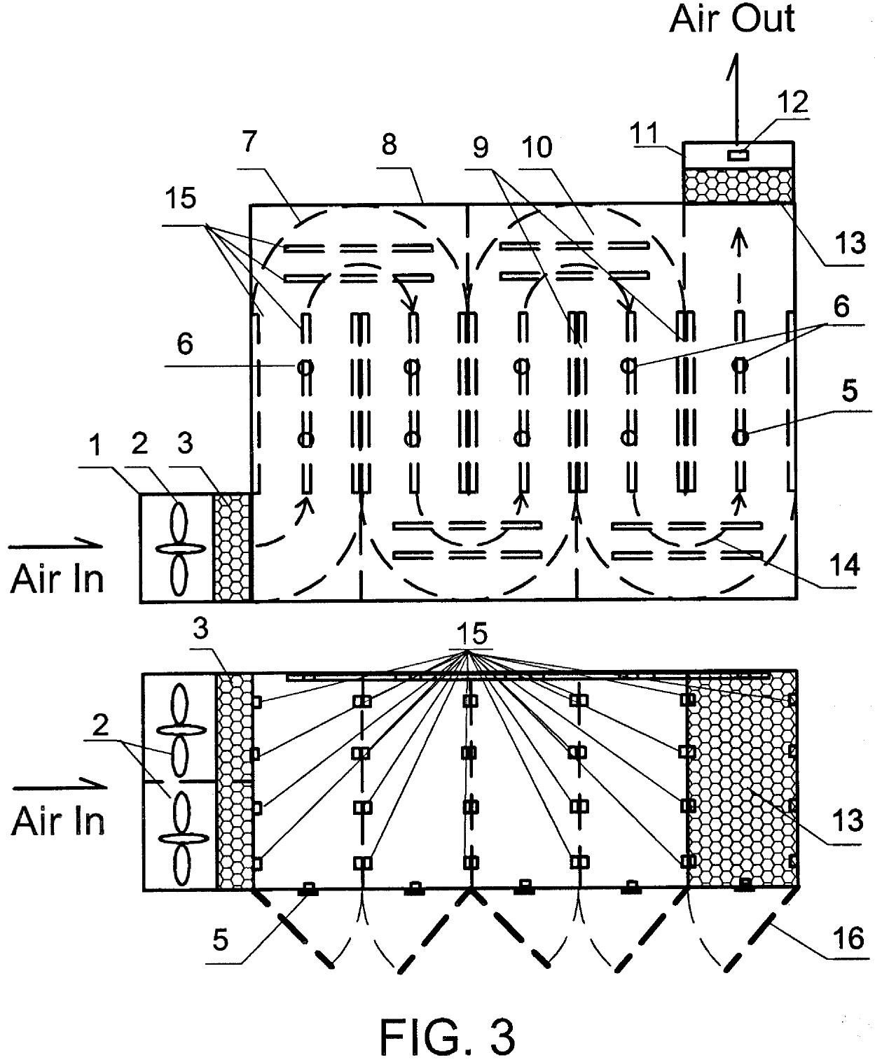

FIG. 3 illustrates an apparatus with a substitution of UV lamps of FIG. 1. In this preferred embodiment of the invention, the UV lamp tubes 15 here are preferably double end tube-shape non-ozone germicidal lamps, lining up in any pattern. The incoming side of the lamp sockets shall be covered with airflow guiding plates (not shown) to reduce airflow resistance. These kinds of UV lamps have the characteristics of higher UV power output and lower cost. In order to perform maintenance, there are five maintenance doors with anti-ultraviolet sealing on the edges in this embodiment.

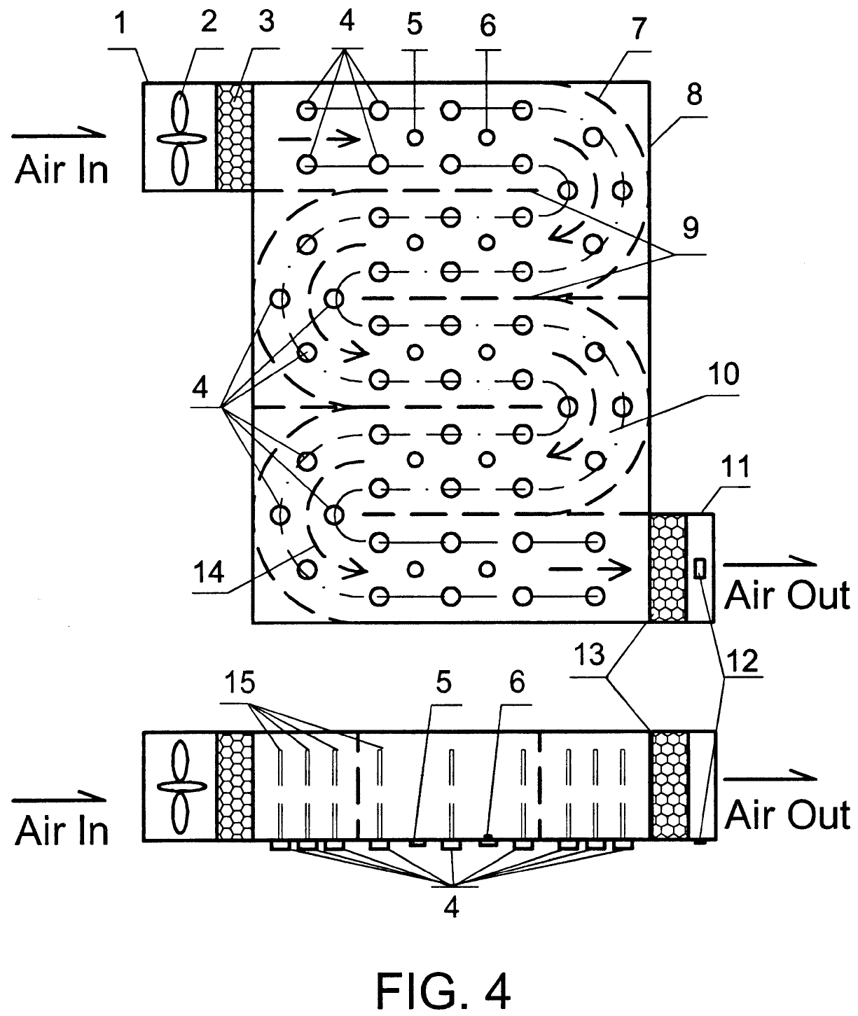

FIG. 4 depicts another preferred embodiment of this invention. This embodiment is with a different orientation. To meet different installation needs, the inlet 1 is preferably positioned on the other side of the air outlet 11 and the UV lamp fixtures 4 only installed on one side of the housing 8.

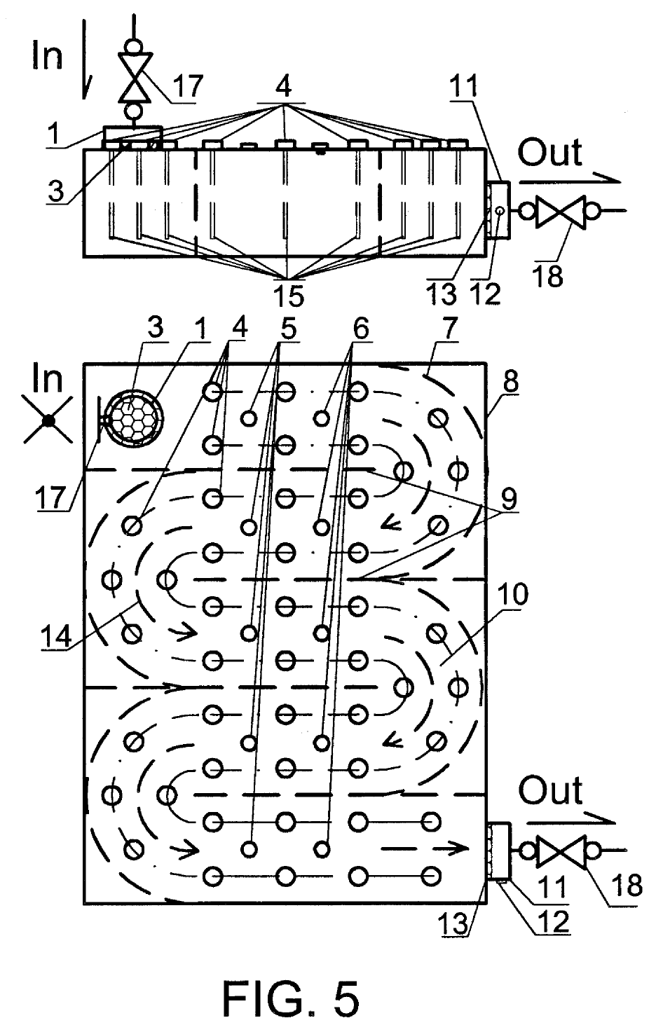

FIG. 5 depicts an apparatus for sterilizing water in large volume by radiation of ultraviolet rays according to a preferred embodiment of this invention. Water goes through the inlet valve 17, inlet 1, and inlet filter unit 3 then into the circuitous sterilizing chamber 10. The distance between any two UV lamps is preferably about 2″ to 6″. The sterilized water discharges through outlet filter unit 13, outlet 11, and then outlet valve 18. There is sample faucet 12 for water quality supervision.

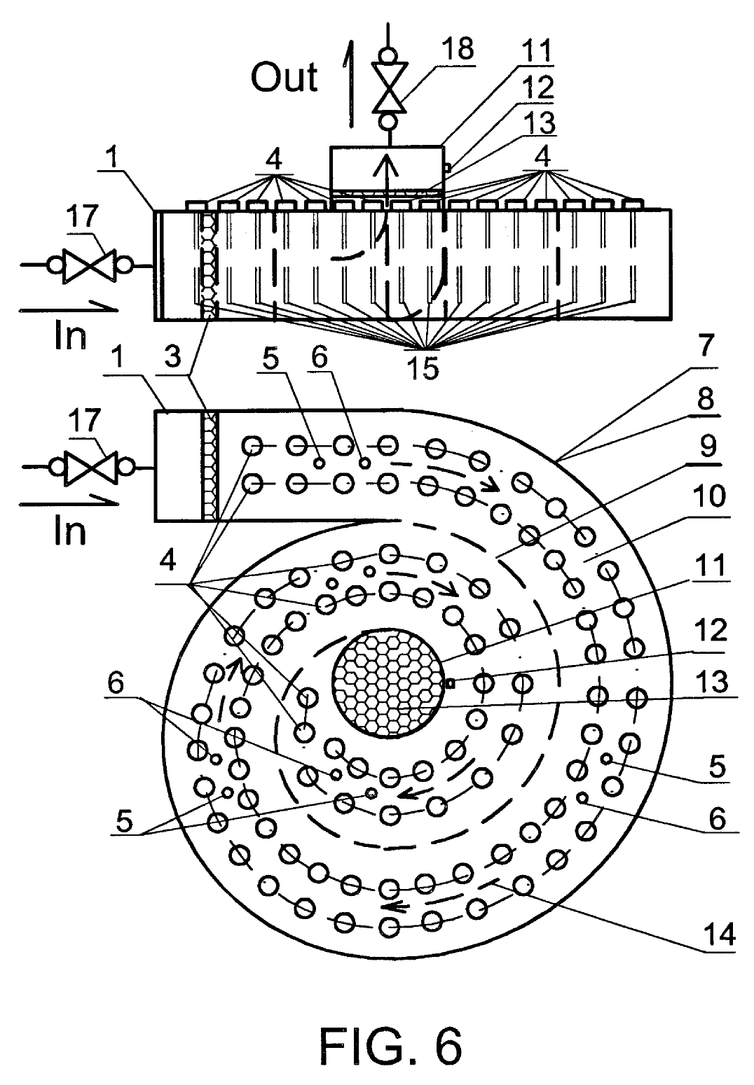

FIG. 6 depicts an apparatus for sterilizing water in large volume by radiation of ultraviolet rays according to another preferred embodiment of this invention. This embodiment constructs a spiral-sterilizing chamber 10, which has lower flow resistance, and more space to install UV lamps. The distance between any two UV lamps is about 2″ to 6″. The sterilized water discharges through outlet filter unit 13, outlet 11, and then outlet valve 18. There is a sample faucet 12 for water quality supervision.

Other alternate embodiments may be devised without departing from the spirit or the scope of the invention. For example, the apparatus depicts in FIG. 6 can be adapted for sterilizing air, and small apparatus can be devised for sterilizing transportation vehicles and residential shelters.

1. A Method for sterilizing fluent material in large volume by radiation of ultraviolet rays, said method comprising the steps of:
(a) guiding and/or forcing fluent material through filter(s) to remove large particles;
(b) using circuitous sterilizing chamber(s) with roundabout path(s), or spiral path(s), or sinuous path(s), or zigzag path(s), or other similar shapes of paths to contain said fluent material;
(c) employing intense UV irradiation to kill all live microorganisms in said fluent material passing through said chamber(s);
(d) converting ozone in air into oxygen when dealing with air;
(e) discharging sterilized fluent material out of said chamber(s).

2. Apparatus for sterilizing fluent material in large volume by radiation of ultraviolet rays, said apparatus comprising:
(a) an inlet 1 guiding in fluent material for sterilizing;
(b) a power unit 2 positioned in said inlet 1;
(c) an inlet filter 3 connected with said inlet 1 to remove fairly large particles from said fluent material;
(d) a circuitous sterilizing chamber 10 connected with said inlet filter unit 3;
(e) a group of UV light tubes 15 positioned, along the flow direction, inside said chamber 10 providing high-density ultraviolet radiation to irradiate passing said fluent material;
(f) connected with said chamber 10, an outlet filter unit 13 to remove any particles larger than the requirements of application;
(g) a catalytic filter comprised in said outlet filter unit 3 to convert ozone into oxygen;
(h) an inspection window or a sample faucet 12 for taking testing samples;
(i) an outlet 11 extending from said outlet filter 13 to discharge sterilized fluent material.

3. The apparatus of claim 2 wherein said circuitous sterilizing chamber 10 may form roundabout path(s), or spiral path(s), or sinuous path(s), or zigzag path(s), or other similar shapes of paths for the purpose of increasing UV exposure.

4. The apparatus of claim 2 wherein said chamber 10 is constructed with smooth curved flow guiding interior 7 at every turning section to form flow low flow resistant chamber.

5. The apparatus of claim 2 wherein said chamber 10 has polished internal reflecting mirror surfaces 9 to increase UV killing effect.

6. The apparatus of claim 2 comprises UV visual inspection window(s) 5 in every section of said chamber 10.

7. The apparatus of claim 2 further comprises UV sensor(s) 6 in every section of said chamber 10 as autofeedback mechanism.

8. The apparatus of claim 2 further comprises an inspection window or a sample faucet 12 on said outlet 11.

9. The apparatus of claim 2 wherein ozone generation is suppressed by use of non-ozone germicidal lamps.

10. The apparatus of claim 2 wherein an outlet filter unit 13 includes a catalytic filter to convert ozone into oxygen when dealing with air.
